(19) United States
(12) Patent Application Publication
(10) Pub. No.: US 2022/0226010 A1
(43) Pub. Date: Jul. 21, 2022

(54) TONGUE SPONGE FOR IMPROVING ORAL CARE

(71) Applicant: ORTHONU, LLC, RUMSON, NJ (US)

(72) Inventors: Sima Yakoby Epstein, Rumson, NJ (US); Douglas Freeman Melville, JR., Simsbury, CT (US)

(21) Appl. No.: 17/504,408

(22) Filed: Oct. 18, 2021

Related U.S. Application Data
(60) Provisional application No. 63/140,247, filed on Jan. 21, 2021.

(51) Int. Cl. A61B 17/24

(52) U.S. Cl. CPC A61B 17/244

(57) ABSTRACT

A tongue sponge for improving oral care includes a wand, a carrier, and a sponge head. The wand has an attachment post positioned at one end thereof. The carrier is for removably and replaceably coupling to the attachment post of the wand. The sponge head has a receptacle for receiving the carrier in a fixed position, with an outwardly facing surface of the sponge extending outwardly around the entire carrier.

### CROSS-REFERENCE TO RELATED APPLICATION

This application claims priority to U.S. Provisional Patent Application No. 63/140,247, filed Jan. 21, 2021, the disclosure of which is incorporated herein by reference in its entirety.

### FIELD

The invention concerns a disposable and/or reusable tongue sponge for improving oral care.

### BACKGROUND

In orthodontic care of patients, there are at least three areas of concern that must be addressed, and which are currently under-addressed in consumer solution products. These include 1) emergency care, 2) oral hygiene, and 3) visual challenges. Emergency care is needed to address discomfort, pain, sensitivity, ulcerations, dry mouth, and the like.

Oral hygiene involves long term oral care and prevention. Oral hygiene includes maintenance care for preventing halitosis and preventing or treating gingivitis and the increase in gram negative bacteria on the tongue and periodontium that are associated with orthodontia care. Avoiding gingivitis and reducing gram negative bacteria in a patient's mouth can prevent and/or deter bone loss and inflammation. This can help to maintain a healthy mouth, tongue, gums, and fresh breath.

Visual challenges include the appearance of white decalcifications on teeth due to the start of cavities that can be attributed to an increase of gram-positive bacteria. Other visible challenges include broken brackets, tooth and elastic discoloration, food stuck between teeth, and staining of enamel.

Gram negative bacteria lives on the tongue and inside the gum line around the periodontium. Gram negative bacteria is a main cause of halitosis in the mouth. Gram positive bacteria is found in the plaque accumulation of the pellicle that forms on the enamel of the tooth that then starts the decalcification process that can lead to cavities, also causing halitosis. Targeting these two types of bacteria with different ingredients and tools and differentiating them can solve many issues inherent in the changes in microbiome during orthodontic treatment.

Patients undergoing orthodontic treatment often undergo multiple adjustments to their braces and/or aligners throughout their treatment. Patients oftentimes use homemade “fixes” when it is impossible to get to their Orthodontists Office. Some examples include eraser heads for broken wires and toenail clippers or wire clippers to cut wires. These options are often not the most hygienic choices and improvements can be made to help the orthodontic patient in dealing with these types of emergencies. Shifting teeth can also trigger increases in bacterial loads. Because of the lack of home care solutions, patients often simply live with the bad breath and plaque that builds up on their teeth. On-the-go solutions and home-care solutions are needed.

### SUMMARY

A tongue sponge is shown and described that incorporate features for improving oral care.

### BRIEF DESCRIPTION OF THE DRAWINGS

FIG. 1 is a perspective view of a tongue sponge according to the invention;

FIG. 2 is a longitudinal cross-sectional view of the tongue sponge of FIG. 1;

FIG. 3 is a top view of the tongue sponge of FIG. 1;

FIG. 4 is a side view of the tongue sponge of FIG. 1;

FIG. 5 is a bottom view of the tongue sponge of FIG. 1;

FIG. 6 is a top view of the tongue sponge of FIG. 1;

FIG. 7 is a cross-sectional view of the tongue sponge head of FIG. 1;

FIG. 8 is an exploded view of the tongue sponge of FIG. 1;

FIG. 9 is a front view of the handle of the tongue sponge of FIG. 1;

FIG. 10 is a side view of the handle shown in FIG. 9;

FIG. 11 is a top view of the handle shown in FIG. 9;

FIG. 12 is a bottom view of the handle shown in FIG. 9;

FIG. 13 is a front view of a connector for connecting the head of the tongue sponge to the handle;

FIG. 14 is a top view of the connector of FIG. 13;

FIG. 15 is a bottom view of the connector of FIG. 13;

FIG. 16 is a cross-sectional side view of the connector of FIG. 13;

FIG. 17 is a side view of the connector of FIG. 13;

FIG. 18 is a front view of the sponge head used with the tongue sponge of FIG. 1;

FIG. 19 is a cross-sectional side view of half of the sponge head shown in FIG. 18;

FIG. 20 is a side view of half of the sponge head shown in FIG. 18;

FIG. 21 is a rear view of half of the sponge head shown in FIG. 18;

FIG. 22 is a perspective view of a tongue sponge according to the invention installed in a stand;

FIG. 23 is a front view of the tongue sponge and stand shown in FIG. 22;

FIG. 24 is a side view of the tongue sponge and stand shown in FIG. 22;

FIG. 25 is a top view of the tongue sponge and stand shown in FIG. 22;

FIG. 26 is a bottom view of the stand shown in FIG. 22;

FIG. 27 is a perspective view of the stand shown in FIG. 22;

FIG. 28 is a front view of the stand shown in FIG. 27;

FIG. 29 is a top view of the stand shown in FIG. 27;

FIG. 30 is a cross-sectional view of the stand shown in FIG. 28 taken along lines 30-30;

FIG. 31 is a bottom view of the stand shown in FIG. 27;

FIG. 32 is a side view of the stand shown in FIG. 27;

FIG. 33 is a rear perspective view of the tongue sponge and stand with holder according to the invention;

FIG. 34 is an exploded perspective view of the tongue sponge and stand with holder as shown in FIG. 33;

FIG. 35 is a side view of the tongue sponge and stand with holder as shown in FIG. 33;

FIG. 36 is a cross-sectional side view of the tongue sponge and stand with holder as taken along line 36-36 in FIG. 37;

FIG. 37 is a front view of the tongue sponge and stand with holder as shown in FIG. 33;

FIG. 38 is a perspective view of the stand shown in FIG. 33;

FIG. 39 is a perspective view of the holder shown in FIG. 33;

FIG. 40 is a front view of the stand shown in FIG. 33;

FIG. 41 is a top view of the stand shown in FIG. 33;

FIG. 42 is a side view of the stand shown in FIG. 33;

FIG. 43 is a cross-sectional side view of the stand shown in FIG. 33 taken along line F-F in FIG. 40;

FIG. 44 is a bottom view of the stand shown in FIG. 33;

FIG. 45 is a front view of the holder shown in FIG. 33;

FIG. 46 is a top view of the holder shown in FIG. 45;

FIG. 47 is a side view of the holder shown in FIG. 45

FIG. 48 is a cross-sectional side view of the holder taken along lines 48-48 in FIG. 45; and

FIG. 49 is a bottom view of the holder of FIG. 45.

### DETAILED DESCRIPTION

Science has shown that most of the bacteria in a patient's mouth resides on the tongue. During orthodontic treatment, patients have challenges with oral hygiene. As teeth shift, the mouth has a greater propensity to harbor bacteria in both the gums and on the tongue. There are known tongue brushes or scrapers that are harsh and can damage the papillae and taste buds on the tongue. Tongue scrapers and brushes often have harsh bristles and abrasive scrubbing action that creates damage to the papillae, opening further micro holes to allow for more bacteria to be accumulated. Existing brushes and scrapers can bruise and/or or damage the tongue, which can exacerbate growth or the harboring of more bacteria over time.

According to the invention, a tongue sponge 10 may avoid the issues with known tongue scrapers and brushes and can be used to help control and remove bacteria that causes gingivitis, inflammation of the periodontium, halitosis, and addresses a myriad of other oral health issues. The tongue sponge 10 is designed such that with one or more swipes around the mouth, including the tongue, it lifts off bacteria from the tongue.

The tongue sponge 10 comprises a system that includes a handle (or wand) 12 and a sponge head 14. An agent may be applied to the sponge head 14 to aid in bacteria removal. The agent may be a liquid, a gel, or another substance. The handle 12 is ergonomic, and the sponge head 14 is small, soft, and positioned at one end of the handle 12. The sponge material can be a loofa or loofa-like. The sponge material can be one or more of: 1) Botanical fibers; 2) Sisal (agave); 3) Biodegradable rayon; 4) Natural vegetable fiber; 5) Nylon; 6) Polyurethane sponge; 7) Natural cellulose fiber; or 8) a combination of any of these materials, among other materials. The sponge head may be made of foam, if desired.

In one embodiment, the agent used with the sponge head 14 is an antibacterial agent, which can be accomplished by pre-loading the sponge head 14 with antibacterial agents. Preloaded antibacterial agents on the sponge head 14 may be activated, for example, by rinsing under water. Alternatively, agents may be activated by coming into contact with saliva. The agent may alternatively be applied at the time of use using a separate antibacterial liquid or gel that can be applied to the sponge head 14 by a user at the time of use. Each sponge head 14 can also include other ingredients, if desired, such as probiotics to boost immunity, anti-virals, vitamins and minerals, or other ingredients, if desired. One example antimicrobial or antibacterial gel may include zinc acetate, a probiotic, and a mint flavoring or extract to eliminate halitosis.

When the patient is ready to use the tongue sponge 10, they either wet the sponge head 14 so that the pre-applied agent is activated, or they apply the agent directly to the sponge head 14. Then the patient rubs the sponge head 14 gently in circular motions on their tongue, slowly massaging bacteria loose from the tongue. The sponge head 14 may also be rubbed gently on the top and bottom gums to eliminate any bacteria that is present.

The tongue sponge 10 of the present invention is an improvement over the prior art because, among other things, it is safer to use because it does not damage the taste buds or papillae of the tongue of a patient. The tongue sponge's 10 spongy, non-abrasive material serves to remove a significant amount of accumulated dead cells and bacteria from the back of a patient's tongue. For example, the tongue sponge 10 may remove approximately about 99% of the white accumulated dead cells and bacteria in the back of a patient's tongue and from the gums.

The tongue sponge 10 has an easy-to-use handle 12 that is small, sleek and ergonomically designed. The handle 12 allows the patient to easily reach all parts of the mouth. The sponge head 14 may be single use or could be reusable. A limitation on usage can also be applied, such as instructing that the sponge head 14 is to be replaced after two uses.

The sponge head 14 may be removable from the handle 12 and can be replaced. The combination of the handle 12 and the sponge head 14 provide a connecting feature that permits the sponge head 14 to attach firmly to the handle 14.

The handle 12 may be reusable, with the sponge head 14 being replaceable, if desired. In this embodiment, the sponge head 14 is removable and replaceable onto the existing handle 12 while the used sponge head 14 is disposed of. Alternatively, the sponge head 14 may be fixed to the handle 12, with the entire tongue sponge 10 being disposable.

A holder 16 can be used with the tongue sponge 10. The holder 16 can be used to prop up the handle 12 and sponge head 14 in an upright position up so that the tongue sponge 10 can dry between uses. The holder 16 can also include a storage compartment 18 that permits the patient to store sponge heads 14 in the holder 16 for later use.

The tongue sponge 10 may have broader uses outside of orthodontia including military personnel applications, hospice, and hospital care, where ensuring that bacteria levels in the mouth are controlled to keep users healthier. The tongue sponge 10 could be used for drug delivery or vitamin/mineral delivery, for example.

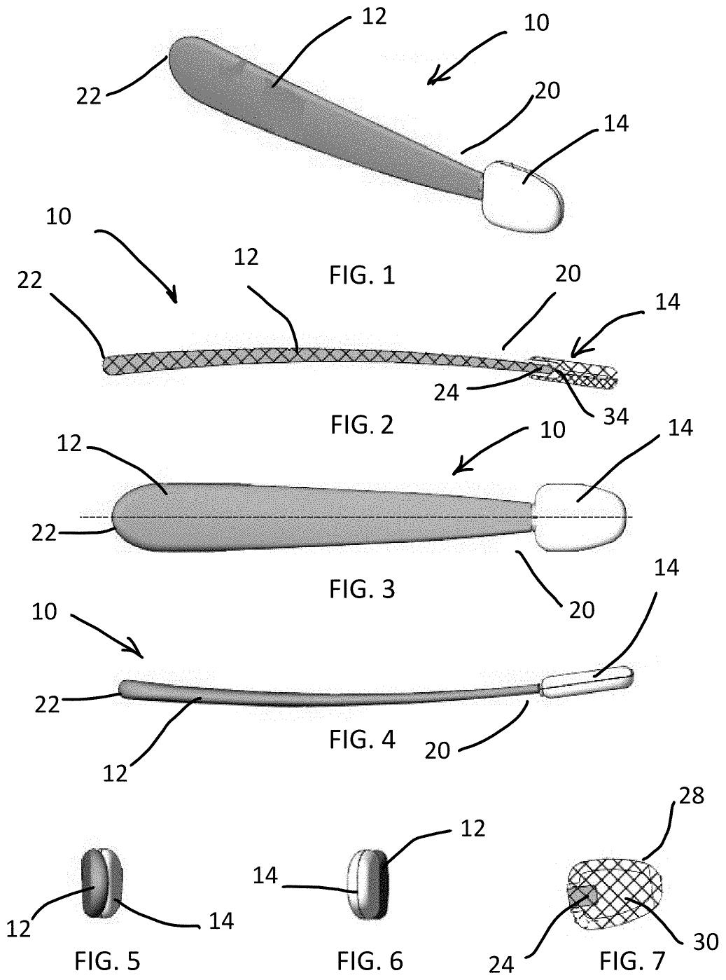

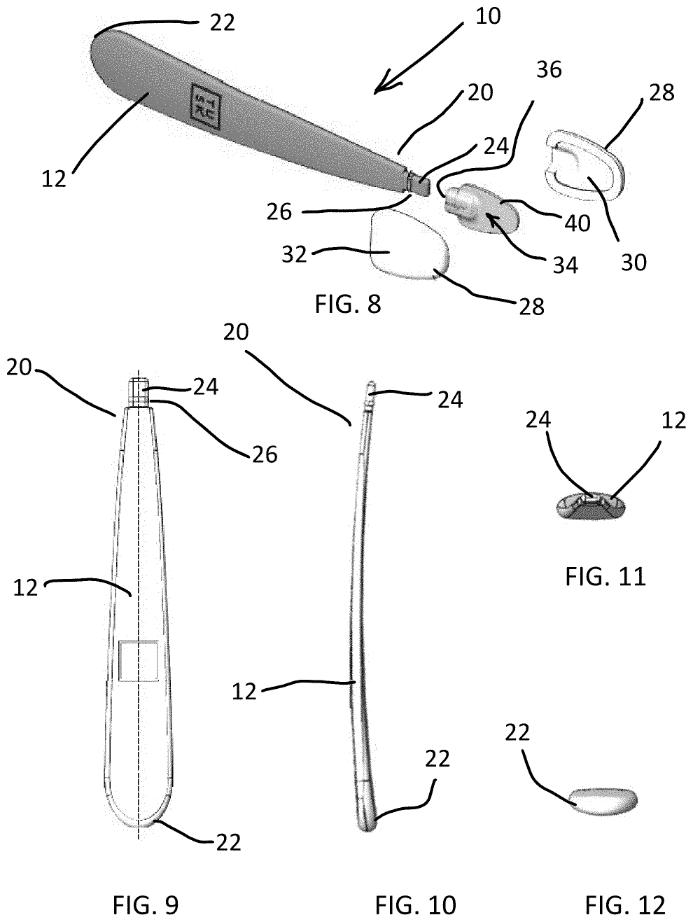

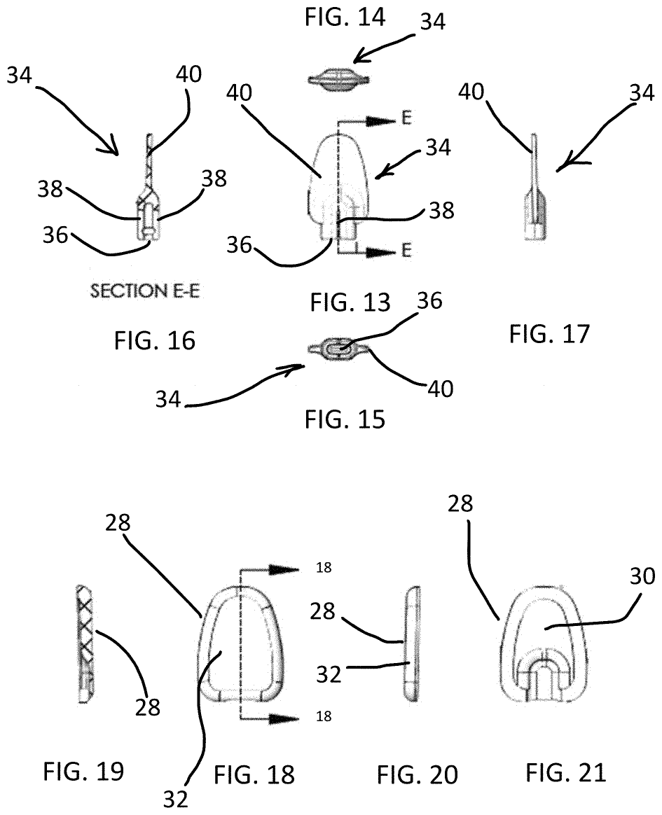

Referring to the drawings, FIGS. 1-21 depict a tongue sponge 10 that includes a handle 12 in the form of a wand 12 and a sponge head 14. The wand 12 serves as a handle for the tongue sponge 10 and the sponge head 14 is the portion of the tongue sponge 10 that is used to clean a patient's tongue. The wand 12 is flattened and curved along its length. The wand 12 is symmetrical about a longitudinal axis X-X from side to side. The wand 12 has a top end 20 and a bottom end 22 and is and curved from top to bottom. A thickness of the wand 12, as shown in FIG. 2, is thicker at the bottom end 22 that at the top end 20. The wand 12 tapers from narrower at the top end 20 to wider at the bottom end 22 in both the front and side dimensions. The wand 12 is curved to easily permit a patient to place the sponge head 14 into their mouth and to be able to effectively engage the patient's gums and tongue.

An attachment member 24 is positioned at the top end 20 of the wand 12 for coupling with the sponge head 14. In one embodiment, the attachment member 24 incorporates a snap feature in the form of a rectangular attachment post that has a recessed ring 26 positioned around the bottom of the attachment post 24. In use, the attachment post 24 is positioned inside the sponge head 14. The exterior surface of the wand 12 may include indicia to indicate the brand of the product, other writing or labels, instructions, or otherwise.

The sponge head 14 is removable and replaceable onto the wand 12. The sponge head 14 includes complementary die cut sponge members 28 that are positioned on top of each other such that an inner surface 30 of each sponge member 28 faces inwardly and an outer surface 32 faces outwardly. The outer surface 32 may be smooth or textured. The outer surface 32 of each sponge member 28 may have an arcuate outer surface, be substantially flat, or be flat with rounded outer edges.

A carrier 34 is positioned between the inner surfaces 30 of the sponge members 28 and is used to firmly attach the sponge members 28 to the wand 12. The carrier 34 includes a receptacle 36 for mating with the attachment head 24 of the wand 12. The receptacle 36 includes a slit 38 along one or both sides thereof that permits the surfaces of the receptacle 36 of the carrier 34 to flex open to permit the attachment post 24 of the wand 12 to enter the interior of the carrier 34. A web 40 extends outwardly from the receptacle 36. The web 40 is used to provide stability to the sponge head 14. The web 40 extends from a top end of the carrier 34 and around the sides of the receptacle 36.

In the embodiment shown, the receptacle 36 of the carrier 34 is for mating with the snap feature of the wand 12, which is provided by the attachment head 24. The outer edges of the sponge members 28 are heat sealed together around the carrier 34 to hold the carrier 34 in position between the sponge members. The carrier 34 may be adhered to the inner surfaces 30 of the sponge members 28 with an adhesive prior to heat sealing or instead of heat sealing, if desired.

In one embodiment, the wand 12 is approximately 5.7 inches long, about ¾ inches wide at the bottom end and about 0.3 inches wide at the top end, with the attachment post 24 being about 0.2 inches wide. The wand 12 can range in size from about 4 inches to about 7 inches and have a width ranging from about 0.5 to 1.5 inches at the bottom end 22 and from about 0.2 inches to about 0.5 inches at the top end 20. Other sizes may also be used.

In one embodiment, the sponge head 14 is about ¾ inches wide and about 1 inch long and the sponge members 28 are about 0.1 inch (3 mm) thick. The sponge head 14 may range in size from about ½″ to 1″ wide, about ½″ to 1.5″ long, and about 0.05 inches to about 0.25 inches thick. Other dimensions may also be used.

In one embodiment, the carrier 34 is about ½″ wide, about 0.9 inches long, about 0.2 inches wide at the bottom end thereof, and about 0.05 inches at the top edge thereof. An interior opening of the receptacle 36 of the carrier 34 may be about 0.2 inches wide to mate with a similarly sized attachment post 24 of the wand 12. A slit 38 may be about 0.3 inches long. Other sizes may also be used for the carrier 34 and will be based upon the size of the sponge head 14.

The tongue sponge 10 may include a holder 16 for holding the tongue sponge 10 in an upright position. In one embodiment, the holder 16 is tray-like and has an opening for holding the bottom end 22 of the wand 12. In another embodiment, the holder 16 has multiple trays, with an upper tray 42 having an opening 44 for holding the bottom end 22 of the wand 12 and a lower separate tray 46 for storing additional sponge heads 14. In the multi-tray embodiment, the lower tray 46 seats inside a downwardly facing receptacle of the upper tray 42 to form a two-part integral member.

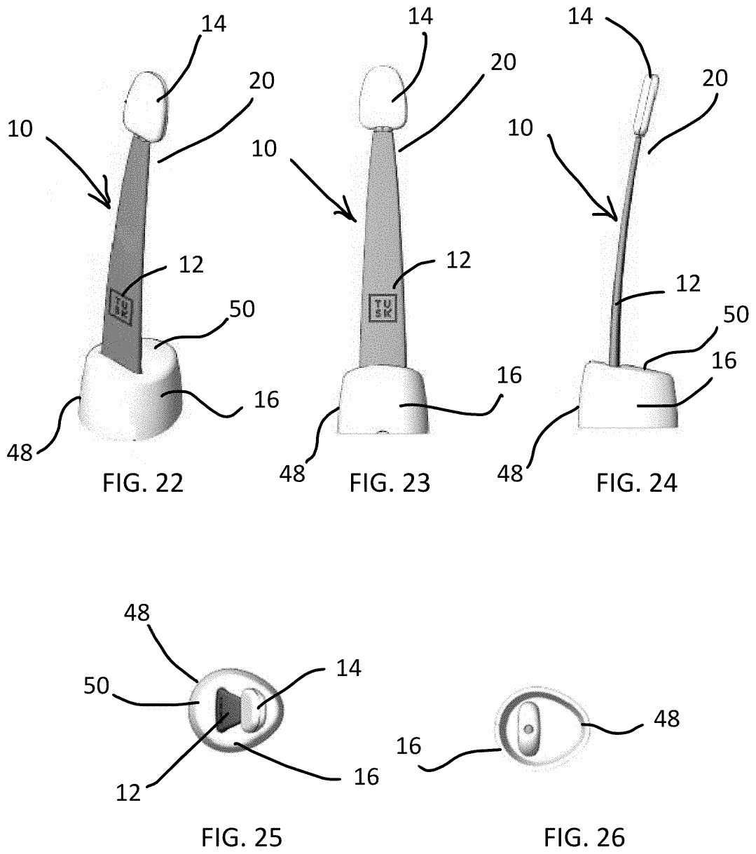

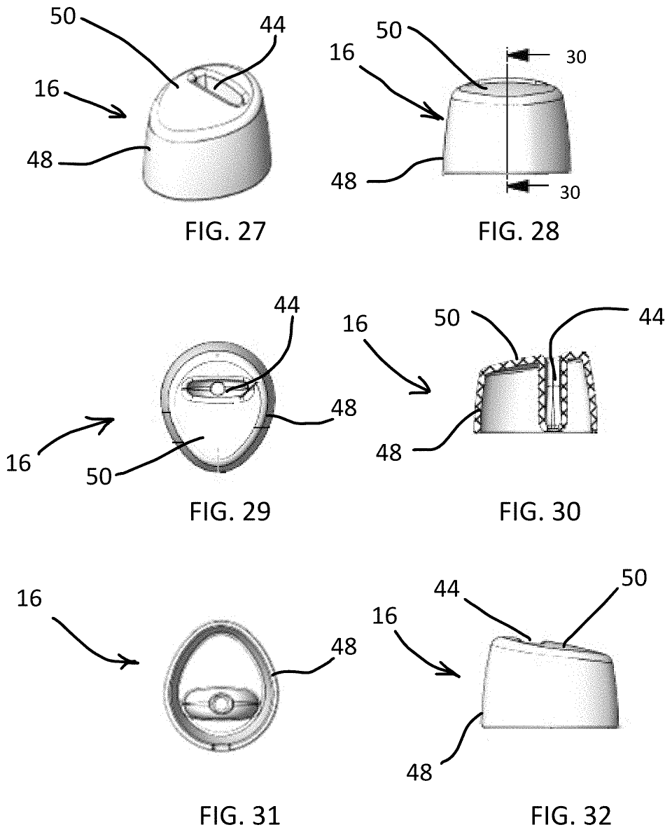

FIGS. 22-32 depict a single tray-like member/holder 16 for holding the tongue sponge 10 wand 12. The holder 16 as shown is approximately oval in shape and has a height sufficient to hold part of the bottom end 22 of the wand 12. The holder 16 can be other shapes, if desired.

The holder 16 includes an outer side wall 48 that extends from a bottom end to a top end, with a top wall 50 provided at the top end, and a downwardly extending recess 44 provided in the top wall 50. The downwardly extending recess 44 is shaped and sized for receiving the bottom end 22 of the wand 12. The recess 44 may be vertical or angled relative to vertical.

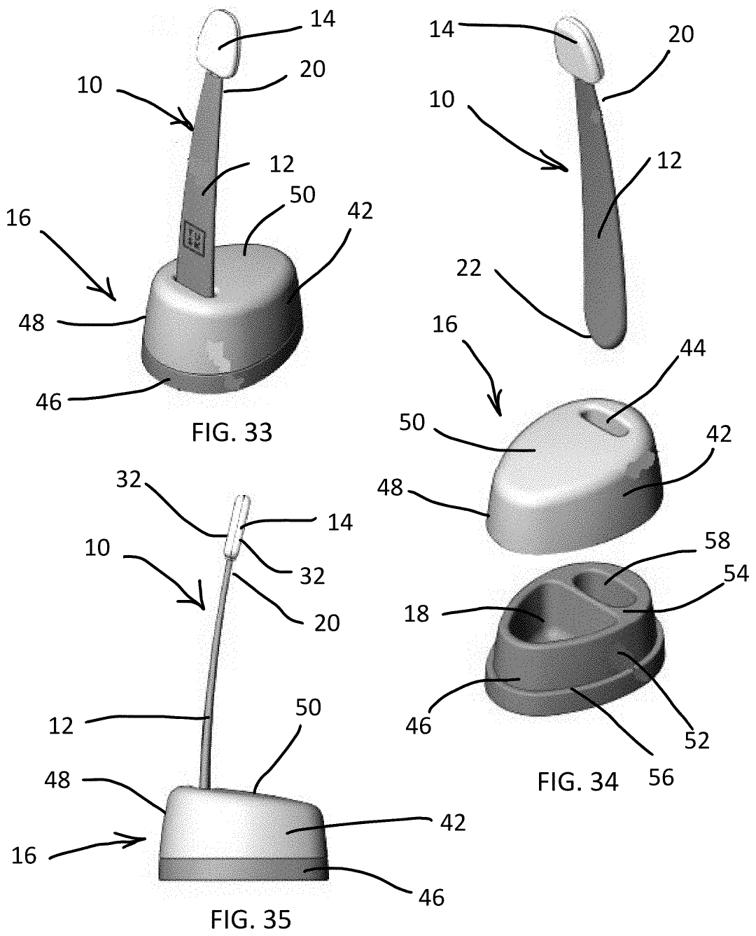

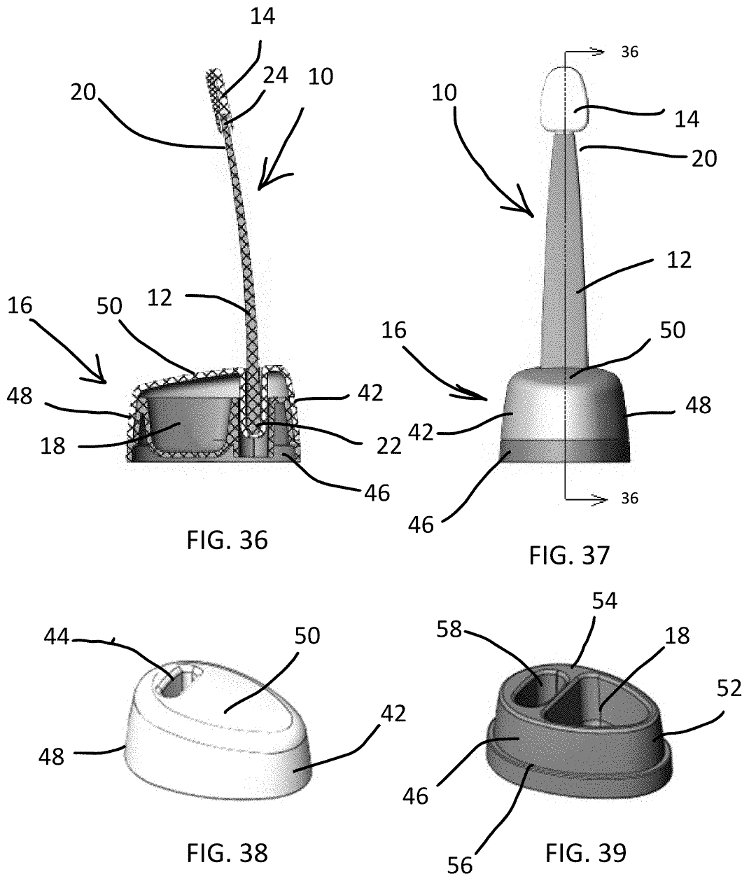

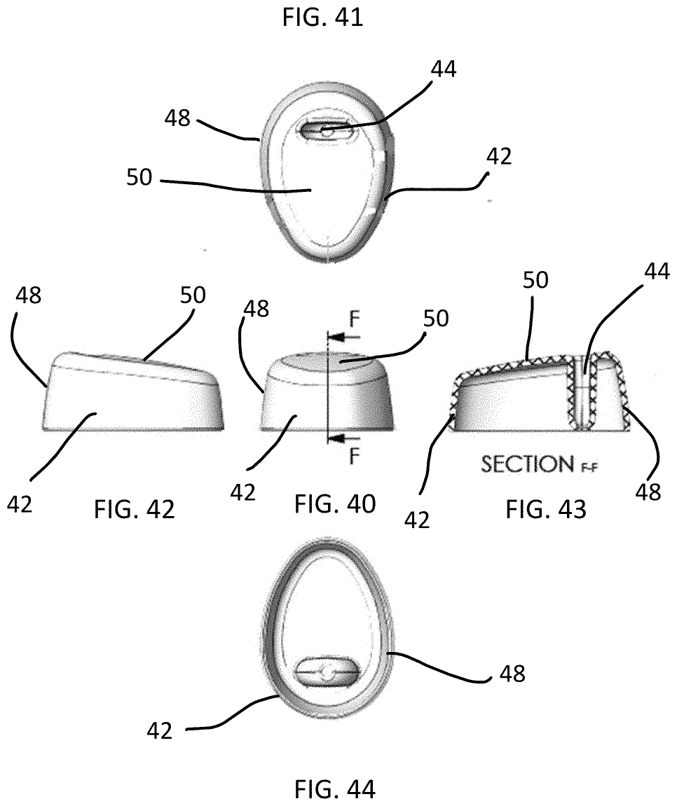

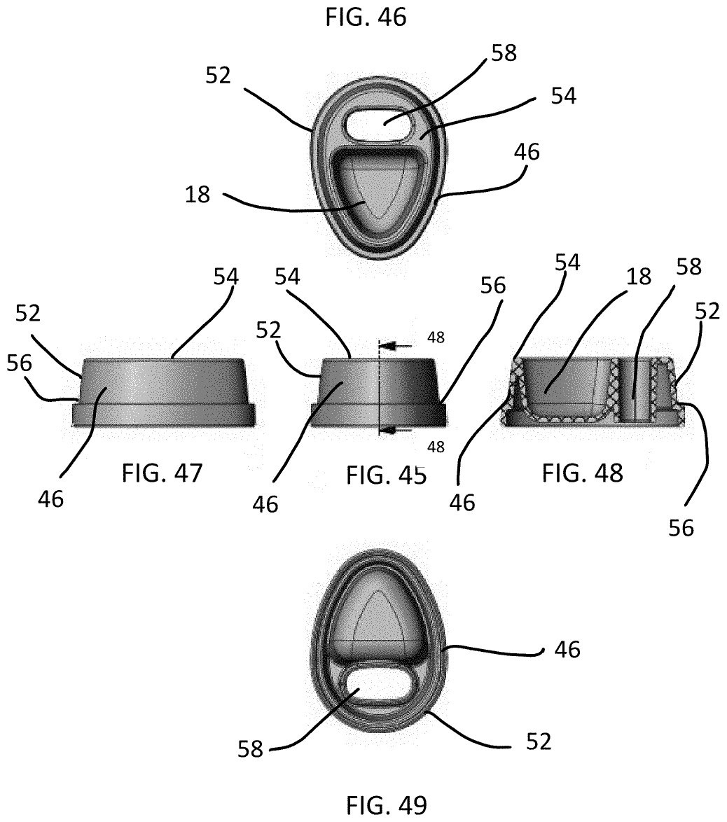

FIGS. 33-49 depict a holder 16 that includes two tray-like members 42, 46 that seat together to provide an integral member for holding the tongue sponge 10. The holder 16 includes an upper tray 42 and a bottom tray 46. The holder 16 is generally oval in shape and includes a side wall and a top wall. The upper tray 42 is similar in shape to the holder 16 shown in FIGS. 22-32 in that it has a side wall 48 with a bottom end and a top end. A top wall 50 is attached to the top end of the side wall 48 of the upper tray 42. The top wall 50 includes a recess 44 for receiving a bottom end 22 of the wand 12. The recess 44 may be oriented vertical or angled. The bottom end of the upper tray 42 may be seated on a surface, such as a tabletop, or on top of the bottom tray 46.

The bottom tray 46 also includes a side wall 52 and a top wall 54. The side wall 52 has a bottom end and a top end, and the top wall 54 is coupled to the top end of the side wall 52. The bottom end is configured to seat on a surface. The side wall 52 of the bottom tray 46 includes a shelf or ledge 56 positioned part way up the side of the side wall 52 for accepting the bottom end of the upper tray 42 and to form a substantially smooth outer wall. The bottom tray 46 includes an upper wall 54 that includes a recessed portion 18 for accepting sponge heads 14. In addition, the bottom tray 46 includes an opening 58 in the top wall 54 of the bottom tray 46 for receiving the recessed portion 44 of the upper tray 42 in which the wand 12 seats. Together, the recess 44 and the opening 58 for receiving the recessed portion of the upper tray 42 take up a majority of the upper wall 54 of the lower tray 46.

In use, the upper tray 42 seats on the bottom tray 46 and the bottom tray 46 seats on a surface. The patient stores sponge heads 14 in the recess 18 of the lower tray 42 and covers the sponge heads 14 with the upper tray 42. The wand 12 is positioned in the recess 44 of the upper tray 42 for storage. When a sponge head 14 needs to be replaced, the patient simply removes the used sponge head 14 from the wand 12, removes a replacement sponge head 14 from the lower tray 46, and positions the new sponge head 14 on the wand 12.

As discussed above, the sponge head 14 may be pre-coated with a solution that can be activated by water or saliva or both. Alternatively, a solution can be applied to the sponge head 14 prior to use. If desired, the holder 16 could be enlarged to provide a space for holding a solution bottle.

In one embodiment, a tongue sponge 10 for improving oral care includes a wand 12, a carrier 34, and a sponge head 14. The wand 12 has an attachment post 24 positioned at one end thereof. The carrier 34 is for removably and replaceably coupling to the attachment post 24. The sponge head 14 has a receptacle or internal cavity 36 for receiving the carrier 34 in a fixed position, with an outwardly facing surface of the sponge head 14 extending outwardly around the entire carrier 34.

The attachment post 24 may be substantially rectangular in shape and may have a recessed channel 26 positioned at a bottom end thereof. The recessed channel 26 permits the carrier 34 to snap around the attachment post 24.

The sponge head 14 may include a first side member 28 and a second side member 28. The carrier 34 may be positioned between the first side member 28 and the second side member 28, with the first and second side members 28 of the sponge head 14 being joined together at least around part of the periphery thereof.

The carrier 34 may have two opposed sides. The carrier 34 may be adhered to the first side member 28 of the sponge head 14 on one side thereof and to the second side member 28 of the sponge head 14 on the other side thereof.

The sponge head 14 may have an outwardly facing surface 32 that comprises two faces 28 that are opposed to one another and are welded together at their periphery. Each face 28 of the sponge head 14 may have an inner surface 30. The inner surface 30 of each sponge face 28 may be adhered to the carrier 34. The carrier 34 may be configured to snap onto the attachment post 24 of the wand 12 and to be removable from the attachment post 24.

The wand 12 may be curved when viewed from the side thereof. The carrier 34 may include a receptable 36 and an outer web 40 extending outwardly from the receptacle 36. The outer web 40 may be sized to seat inside the sponge head 14.

The receptacle 36 of the carrier 34 may have at least one longitudinally extending slit 38 therethrough that permits the carrier 34 to flex around the attachment post 24. The sponge head 14 may include an oral care solution.

In another embodiment, an apparatus for improving oral care includes a wand 12, a dual-faced sponge head 14, and a carrier 34. The wand 12 has a length and a width, with an attachment head 24 positioned at one end thereof. The dual faced sponge head 14 includes a first sponge side 28 having a first outwardly facing surface 32, a first inner surface 30, and a first outer periphery. The dual faced sponge head 14 also includes a second sponge side 28 having a second outwardly facing surface 32, a second inner surface 30, and a second outer periphery. The first and second sides 28 of the sponge are joined around at least part of the first and second outer peripheries and define a cavity between the first and second sponge sides. The carrier 34 has a receptacle 36 for mating with the attachment head 24 of the wand 12 that is seated between the first and second sponge sides 28. The carrier 34 holds the sponge head 14 on the wand 12.

The carrier 34 may be sized to seat in the cavity between the first and second sponge sides 28. The first sponge side 28 may be welded to the second sponge side 28. The first sponge side 28 may have substantially the same size as the second sponge side 28.

The carrier 34 may be adhered to the first and second sponge sides 28 in the cavity. The carrier 34 may have a web 40 extending outwardly from the receptacle 36 and the web 40 may be a strengthening member for the sponge head 14. The carrier 34 may be configured to snap around the attachment head 24. The carrier 34 may be removable from the attachment head 24.

The sponge head 14 may receive an oral care solution. The sponge head 14 may be coated with an antibacterial solution that is activated when wetted.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. All percentages and averages are by weight unless the context indicates otherwise. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A tongue sponge for improving oral care comprising:
a wand having an attachment post positioned at one end thereof;
a carrier for removably and replaceably coupling to the attachment post; and
a sponge head having a receptacle for receiving the carrier in a fixed position, with an outwardly facing surface of the sponge head extending outwardly around the entire carrier.

2. The tongue sponge of claim 1, wherein the attachment post is substantially rectangular in shape and has a recessed channel positioned at a bottom end thereof, and the recessed channel permits the carrier to snap around the attachment post.

3. The tongue sponge of claim 1, wherein the sponge head comprises a first side member and a second side member, and the carrier is positioned between the first side member and the second side member, with the first and second side members of the sponge head being joined together at least part of the periphery thereof.

4. The tongue sponge of claim 3, wherein the carrier has two opposed sides, and the carrier is adhered to the first side member of the sponge head on one side thereof and to the second side member of the sponge head on the other side thereof.

5. The tongue sponge of claim 1, wherein the sponge head outwardly facing surface comprises two faces that are opposed to one another and are welded together at their periphery.

6. The tongue sponge of claim 5, wherein each face of the sponge head has an inner surface, and the inner surface of each sponge face is adhered to the carrier.

7. The tongue sponge of claim 1, wherein the carrier is configured to snap onto the attachment post of the wand and to be removable from the attachment post.

8. The tongue sponge of claim 1, wherein the wand is curved when viewed from the side thereof.

9. The tongue sponge of claim 1, wherein the carrier includes a receptable and an outer web extending outwardly from the receptacle, and the outer web is sized to seat inside the sponge head.

10. The tongue sponge of claim 9, wherein the receptacle of the carrier has at least one longitudinally extending slit therethrough that permits the carrier to flex around the attachment post.

11. The tongue sponge of claim 1, wherein the sponge head includes an oral care solution.

12. An apparatus for improving oral care comprising:
a wand having a length and a width, with an attachment head positioned at one end thereof;
a dual faced sponge head comprising a first sponge side having a first outwardly facing surface, a first inner surface, and a first outer periphery; and a second sponge side having a second outwardly facing surface, a second inner surface, and a second outer periphery, with the first and second sides of the sponge being joined around at least part of the first and second outer peripheries and defining a cavity between the first and second sponge sides; and
a carrier having a receptacle for mating with the attachment head of the wand and seated between the first and second sponge sides, said carrier holding the sponge head on the wand.

13. The apparatus of claim 12, wherein the carrier is sized to seat in the cavity between the first and second sponge sides, and the first sponge side is welded to the second sponge side.

14. The apparatus of claim 12, wherein the first sponge side has substantially the same size as the second sponge side.

15. The apparatus of claim 12, wherein the carrier is adhered to the first and second sponge sides.

16. The apparatus of claim 12, wherein the carrier has a web extending outwardly from the receptacle and the web is a strengthening member for the sponge head.

17. The apparatus of claim 12, wherein the carrier is configured to snap around the attachment head.

18. The apparatus of claim 12, wherein the carrier is removable from the attachment head.

19. The apparatus of claim 12, wherein the sponge head receives an oral care solution.

20. The apparatus of claim 12, wherein the sponge head is coated with an antibacterial solution that is activated when wetted.
